(19) United States
(12) Patent Application Publication
(10) Pub. No.: US 2024/0424077 A1
(43) Pub. Date: Dec. 26, 2024

(54) A METHOD TO PRODUCE A VACCINE AGAINST STREPTOCOCCUS SUIS AND THE SAID VACCINE

(71) Applicant: Intervet Inc., Rahway, NJ (US)

(72) Inventors: Antonius Arnoldus Christiaan Jacobs, Kessel (NL); Remco Bron, Nijmegen (NL)

(73) Assignee: Intervet Inc., Rahway, NJ (US)

(21) Appl. No.: 18/708,248

(22) PCT Filed: Nov. 17, 2022
(86) PCT No.: PCT/EP2022/082177

§ 371 Date: May 8, 2024

(30) Foreign Application Priority Data
Nov. 18, 2021 (EP) 21208911.4

(51) Int. Cl. A61K 39/09; A61K 39/00; A61P 31/04; C12N 1/06

(52) U.S. Cl. CPC A61K 39/092; A61P 31/04; C12N 1/066; A61K 2039/552; A61K 2039/575

(57) ABSTRACT

The invention pertains to a method to produce a vaccine to protect a pig against a pathogenic infection with Streptococcus suis, the method comprising recombinantly expressing an IgM protease antigen in E. coli bacteria, subjecting the E. coli bacteria to a high pressure homogenisation operation at a pressure of at least 500 bar to induce lysis of the E. coli bacteria and release of the IgM protease antigen into the supernatant of the lysate, separating the supernatant from the pellet and mixing the supernatant comprising the IgM protease antigen with a pharmaceutically acceptable carrier to constitute the vaccine. The invention also pertains to a vaccine produced with this method.

### GENERAL FIELD OF THE INVENTION

The invention in general pertains to a method of producing a vaccine for the protection of pigs against a pathogenic infection with Streptococcus suis bacteria, the vaccine being based on an IgM protease antigen of Streptococcus suis, recombinantly expressed in E. coli bacteria.

### BACKGROUND OF THE INVENTION

Streptococcus suis (S. suis) is one of the principal etiologic agents of contagious bacterial disease in pigs. The pathogen can cause a variety of clinical syndromes including meningitis, arthritis, pericarditis, polyserositis, septicaemia, pneumonia and sudden death. S. suis is a gram-positive facultatively anaerobic coccus, originally defined as Lancefield groups R, S, R/S or T. Later, a new typing system based on the type-specific capsular polysaccharide antigens located in the cell wall was proposed. This led to a system comprising 35 serotypes (Rasmussen and Andresen, 1998, “16S rDNA sequence variations of some Streptococcus suis serotypes”, Int. J. Syst. Bacteriol. 48, 1063-1065) of which serotypes 1, 2, 7 and 9 are currently the most prevalent, especially in Europe. However, it is recognised that the capsular serotype is a poor marker of virulence. Therefore, an alternative system to helping understand the epidemiology of S. suis infection and the biological relevance of the serotyping approach was developed, i.e. the so called multilocus sequence typing (MLST), as described by King et al. in the Journal of Clinical Microbiology, October 2002, p. 3671-3680 (Development of a Multilocus Sequence Typing Scheme for the pig pathogen Streptococcus suis: Identification of virulent clones and potential capsular serotype exchange”). In that study 92 sequence types were identified, of which ST complexes ST1, ST27 and ST87, each containing multiple sequence types, dominate the population. See also the Streptococcus suis MLST website (https://pubmlst.org/ssuis/) sited at the University of Oxford (Jolley et al. Wellcome Open Res 2018, 3:124 (site funded by the Wellcome Trust), which refers to the King et al. paper and allows for easy identification of the sequence type for any Streptococcus suis strain.

Control of Streptococcus suis in pig herds appears to be difficult. Streptococcus suis is a commensal and opportunistic pathogen of swine. Apparently, the immune system is not triggered in each and every occasion of an infection. Next to this, Streptococcus suis is a well-encapsulated pathogen and uses an arsenal of virulence factors to evade the host immune system. Together, these characteristics have challenged the development of efficacious vaccines to fight this important pathogen. An overview article has been published a few years ago, the article reviewing existing and explorative vaccines against Streptococcus suis (Mariela Segura: “Streptococcus suis vaccines: candidate antigens and progress, in Expert Review of Vaccines, Volume 14, 2015, Issue 12, pages 1587-1608). In this review, clinical field information and experimental data have been compiled and compared to give an overview of the status of vaccine development against Streptococcus suis.

In the last couple of years, an extensive list of antigenic or immunogenic Streptococcus suis molecules has been reported, and most of these have been discovered through immuno proteomics using either convalescent sera from infected pigs or humans and/or laboratory-produced immune sera. WO2015/181356 (IDT Biologika GmbH) has shown that IgM protease antigens (either the whole protein or the highly conserved Mac-1 domain representing only about 35% of the full protein) can elicit a protective immune response in piglets through vaccination with the IgM protease antigen, optionally in combination with a prime vaccination containing a bacterin. It is suggested in the '356 patent application that the IgM protease antigen, due to the fact that it is highly conserved throughout most if not all Streptococcus suis serotypes, in particular the most prevalent serotypes 1, 2, 7 and 9, that the IgM protease antigen can be used to arrive at broad cross protection among Streptococcus suis serotypes, in particular among serotypes 1, 2, 7 and 9. WO2017/005913 (Intervacc AB) confirms the fact that the IgM protease is highly conserved throughout various Streptococcus suis serotypes.

In the art, the IgM protease antigen is produced using the technique as published by Seele et al. in the Journal of Bacteriology p. 930-940, March 2013, Volume 195, Number 5 (“Identification of a Novel Host-Specific IgM Protease in Streptococcus Suis”). In this technique, the IgM protease antigen is recombinantly expressed in E. coli bacteria and purified by Ni²⁺-nitrilotriacetic acid affinity chromatography under native conditions. This way the IgM protease antigen can be obtained in a highly purified form, however at a relatively low yield. The purified antigen can be mixed with a pharmaceutically acceptable carrier to constitute a vaccine to protect against a pathogenic infection with Streptococcus suis.

### OBJECT OF THE INVENTION

It is an object of the invention to provide an alternative method to produce a vaccine comprising an IgM protease antigen.

### SUMMARY OF THE INVENTION

In order to meet the object of the invention a method to produce a vaccine as described here above in the GENERAL FIELD OF THE INVENTION section has been devised, the method comprising recombinantly expressing an IgM protease antigen of Streptococcus suis in E. coli bacteria, subjecting the E. coli bacteria to a high pressure homogenisation operation at a pressure of at least 500 bar to induce lysis of the E. coli bacteria and release of the IgM protease antigen into the supernatant of the obtained lysate, separating the supernatant from the pellet of the lysate and mixing the supernatant comprising the IgM protease antigen with a pharmaceutically acceptable carrier to constitute the vaccine.

It was found that for obtaining high amounts of the IgM protease, cell lysis of the E. coli bacteria is necessary. Apparently, the IgM protease antigen is not released by the bacteria into the supernatant. However, it was also found that the cells must undergo a high pressure homogenisation operation at a pressure of at least 500 bar to make sure adequate amounts of the antigen are actually released into the supernatant, instead of remaining bound to remnants of the E. coli cells. Releasing high amounts of the antigen into the supernatant has the important advantage that a vaccine can be very simply formulated by using this supernatant directly, not comprising (substantial amounts of) cell debris, optionally after filtration, further clarification, inactivation, concentration etc. as the source of the antigen, not needing high affinity purification over a column. Although such (or other) purification may be done, it was found that the presence of other proteins and small molecules originating from the E. coli bacteria themselves, are not a principal problem for safety and efficacy of the vaccine. Thus, a purification step may be dispensed with when using the method of the present invention.

The present method is easy to perform, results in high yields, and an adequate vaccine. The invention is also embodied in a vaccine comprising an IgM protease antigen of Streptococcus suis obtained using this method. This vaccine differs from the known vaccine in that a substantial amount of E. coli proteins (in particular more than 5%, 10%, 15%, 20%, 25%, 30%, 50% or even a larger percentage of the total amount, i.e. weight, of protein) and other E. coli compounds (e.g. polysaccharides) may be present in the vaccine.

### Definitions

A vaccine is a constitution suitable for application to a subject, comprising one or more antigens in an immunologically effective amount (i.e. capable of stimulating the immune system of the target subject sufficiently to at least reduce the negative effects of a challenge of the wild-type micro-organisms), typically combined with a pharmaceutically acceptable carrier, which upon administration to the subject induces an immune response for treating an infection, i.e. aiding in preventing, ameliorating or curing the infection or any disease or disorder arising from that infection.

Protection against a pathogenic infection with a micro-organism is the same as arriving at protective immunity, i.e. aiding in preventing, ameliorating or curing the pathogenic infection with that micro-organism or a disorder arising from that infection, for example to prevent or reduce of the actual infection or one or more clinical signs resulting from the pathogenic infection with the pathogen.

An IgM protease antigen of Streptococcus suis is an enzyme that specifically degrades porcine IgM (and not porcine IgG or porcine IgA; Seele at al, in Journal of Bacteriology, 2013, 195 930-940; and in Vaccine 33:2207-2212; 5 May 2015), a protein denoted as IdeSsuis, or an immunogenic part thereof (typically having a length of at least about 30-35% of the full length enzyme). The whole enzyme has a weight of about 100-125 kDa, corresponding to about 1000-1150 amino acids, the size depending on the serotype of S. suis. In WO 2015/181356 several sequences that represent an IgM protease antigen of Streptococcus suis are given, viz. SEQ ID NO:1, SEQ ID NO:2, SEQ ID NO:6, SEQ ID NO: 7 and SEQ ID NO:5, the latter being an immunogenic part of the full length enzyme (denoted as the Mac-1 domain, i.e. amino acids 80-414 of SED ID NO: 7). Other examples of immunogenic parts of the full length enzyme are given in WO2017/005913. In particular the IgM protease may be the protease according to SEQ ID NO: 1 of WO2015/1818356 or a protein having at least 70%, in particular 75, 80, 81, 82, 83, 84, 85, 86, 87, 88, 89, 90, 91, 92, 93, 94, 95, 96, 97, 98, 99% up to 100% sequence identity in the overlapping regions. The amino acid sequence identity may be established with the BLAST program using the blastp algorithm with default parameters. It is expected that the IgM proteases of Streptococcus suis of various serotypes have a sequence identity higher than 70%, in particular expected to be 75, 80, 81, 82, 83, 84, 85, 86, 87, 88, 89, 90, 91, 92, 93, 94, 95, 96, 97, 98, 99% up to 100%. An artificial protein, for example made to optimize yield in a recombinant production system of the antigen, may lead to a lower amino acid sequence identity such as 85%, 80%, 75%, 70% or even 60% compared with the whole enzyme, while maintaining the required immunogenic function, and is understood to be an IgM protease antigen of Streptococcus suis in the sense of the present invention.

A pharmaceutically acceptable carrier is a biocompatible medium, viz. a medium that after administration does not induce significant adverse reactions in the treated subject, capable of presenting the antigen to the immune system of the subject after administration of the composition comprising the carrier. Such a pharmaceutically acceptable carrier may for example be a liquid containing water and/or any other biocompatible solvent or a solid carrier such as commonly used to obtain freeze-dried vaccines (based on sugars and/or proteins), optionally comprising immunostimulating agents (adjuvants). Optionally other substances such as stabilisers, viscosity modifiers, adjuvants, or other components are added depending on the intended use or required properties of the corresponding vaccine.

A supernatant is the liquid lying above a solid residue after crystallization, precipitation, centrifugation, or other process.

A pellet is an insoluble sediment, for example after centrifugation or other sedimentation method, in a liquid.

High pressure homogenization is a mechanical operation wherein a liquid is pushed with high pressure through a narrow gap (typically in the micrometer range), thereby creating an acceleration in the liquid over a very short distance and by such establishing high shear stress, for example to reduce particle size or to lyse cells. Typical pressures use are 100-2000 bar (Dumont et al in the International Journal of Pharmaceutics 541 (2018) 117-135). The higher the amount of energy applied during the homogenisation process, the smaller the particle size or the more complete the cell lysis.

Microfluidisation is a form of high pressure homogenization that works by passing a liquid through micro channels into an interaction chamber to produce two fine jets that are directed against each other at right angle at high pressures up to typically 2000 bar. As the two microstreams collide, there is a sudden pressure drop and homogenisation occurs as a result of turbulence, cavitation and shear effects that occur on impact. An increase in number of passes through the microfluidizer improves homogenisation, but there is little to be gained by having more than three passes. Devices for microfluidisation can be obtained from Microfluidics™, Westwood, MA, USA.

A French pressure cell press (also called a “French Press”) is an apparatus used in biological experimentation to disrupt the plasma membrane of cells by passing them through a narrow valve under high pressure. It is capable of disrupting cell walls while leaving the cell nucleus undisturbed. The French press was invented by Charles Stacy French of the Carnegie Institution of Washington. The press uses an external hydraulic pump to drive a piston within a larger cylinder that contains the liquid sample. The highly pressurized sample is then squeezed past a needle valve. As the sample passes through the valve, the fluid experiences shear stress and decompression, causing cellular disruption.

A whole IgM protease antigen of Streptococcus suis is an antigen that comprises at least the Mac-1 domain, the region linked to structural functions, the CNV region, and optionally the cell adhesion region (see Example 1 for the identification of these regions in the genome of Streptococcus suis). This can be regarded as a whole IgM protease antigen since the signal peptide is believed to be missing in the naturally occurring (i.e. wild type) secreted enzyme anyway, and the cell adhesion region is not believed to be essential for its function as a protease.

### FURTHER EMBODIMENTS OF THE INVENTION

In a first further embodiment of the method of the invention, the pressure during the high pressure homogenisation operation is at least 1000 bar. This way, more of the IgM protease antigen is released into the supernatant. Preferably, the pressure during the high pressure homogenisation operation is at least 1300 bar, such as 1400, 1500, 1600, 1700, 1800, 1900 or even at least 2000 bar.

In another embodiment of the method according to the invention a device used for performing the high pressure homogenisation operation is a French pressure cell press or a Microfluidisation device. These devices were found to be particularly suitable to perform a method according to the invention. Preferably, a Microfluidisation device is used.

In again another embodiment of the method according to the invention the IgM protease antigen is a whole IgM protease antigen. It was found that this type of antigen, comprising at least the Mac-1 domain, the region linked to structural functions, the CNV region, and optionally the cell adhesion region (see Example 1 for the identification of these regions in the genome of Streptococcus suis), could be expressed at high levels, was easy to get released into the supernatant and very suitable as vaccine antigen. Preferably the whole IgM protease antigen is of a Streptococcus suis bacterium of serotype 1, 2 or 7. Although the recombinant expression for the antigen as such does not depend on the serotype, it was found that antigen of these three serotypes provides adequate protection throughout various serotypes of Streptococcus suis. For this we refer to European patent application EP21189283.1, filed on Aug. 3, 2021 as a priority application with the European Patent Office, in the name of Intervet International BV, titled “A vaccine for protection against Streptococcus suis of various serotypes”.

The invention will now be further explained using the following specific examples.

### EXAMPLES

Example 1: structural analysis of the genome of Streptococcus suis.

Example 2: production of a vaccine comprising whole IgM protease antigen.

Example 3: protective effect of the vaccine.

### Example 1

In this example an analysis of the genome of Streptococcus suis is provided, i.e. the part that encodes for the IgM protease, in order to show how this part of the genome is structured. For this we use the genome of Streptococcus suis of a serotype 2 bacterium, as known from WO 2015/181356, published as SEQ ID NO: 1 in that patent application. The sequence is enclosed again in the sequence listing of the present patent as SEQ ID NO: 1. Sequence similarity search using Needleman-Wunsch alignment (see Needleman et al 1970, Laskowski et al 1997, Apweiler et al 2000; default settings) in addition to protein annotation (PDBSum and InterPro), reveals a structure for the IgM protease genome in which 5 regions can be identified:
Region 1 (Met 1-Thr 34): a signal sequence from position 1;
Region 2 (Val 35-Glu 426): the Mac-1 domain with predicted hydrolase activity;
Region 3 (Thr 427-Pro 687): a region that is linked to structural functions (e.g. involved in proper folding) and substrate binding.
Region 4 (Thr 688-Ser 919): a region that consists of 4 repeats (1*{Thr 688-Ser 744}, 2*{Thr 745-Ser 801}, 3*{Thr 802-Ser 858}, 4*{Thr 859-Ser 919}) that have similarities to known protein sequences with hydrolase activity; This is the so called CNV region (a Copy Number Variation region) in which sections of the genome are repeated;
Region 5 (Thr 920-Lys 1141): contains a predicted transmembrane region indicating cell wall anchor function (the cell adhesion region).

The structure for Streptococcus suis bacteria of other serotypes is largely the same, the most prominent difference being the number of repeats in the CNV region.

### Example 2

In this example a method for producing a vaccine comprising an IgM protease antigen is shown. The whole IgM protease genes of a Streptococcus suis strain of serotype 2 and of serotype 7 were cloned into Escherichia coli (E. coli) using a method as described in Seele et al. (2013; see here above) with the BL21-AI (DE3) plasmid. As inducer arabinose in combination with lactose was used.

E. coli cells were cultivated in an animal component free medium containing lactose, glucose, glycerol, yeast extract, yeastolate, NaCl, KH₂PO₄ and Na₂HPO4×2H₂O. First a pre-culture in a shake flask, gently shaken at 37° C., was used to grow the cells into the middle of the exponential growth phase. Next the cells were inoculated with 1% inoculum ratio into a laboratory scale fermenter of 15 L cultivation scale using the same medium. The pH was controlled at 7.0 using 4 M NaOH and 4 M acetic acid solution. Dissolved oxygen (pO2) was controlled at 50% using cascade control of stirrer speed, airflow and pure oxygen. The temperature was controlled at 37° C. As soon as the glucose in the medium became depleted (determined using a CEDEX analyser) arabinose was added as inducer. The cultivation was continued for 3 more hours to obtain adequate IgM protease protein concentration.

After this, the E. coli cells were collected from the culture and were stored cold either unconcentrated in medium or in 0.04 M PBS buffer, or concentrated up to 4 times using centrifugation. Thereafter cells were disrupted using either a Microfluidizer device (Microfluidics) at 30.000 PSI (2068 bar) or a French press homogenizer (range of 600-2000 bar). Finally, the total fraction after cell disruption was centrifuged and the supernatant and pellet were separated and inactivated using BPL (beta propiolactone). Protein yields were determined using SDS gels with a known BSA series as a reference. The IgM protease was not further purified.

To investigate the effect of pressure during cell disruption, several experiments were performed in which the E. coli cells (serotype 2) were disrupted in either the Microfluidizer or French Press at different pressures. Table 1 shows the relationship between the applied pressure during cell disruption and the recovery of the IgM protease protein in the supernatant after centrifugation after cell disruption. The recovery is calculated by dividing the IgM protease protein concentration in the supernatant after cell disruption and centrifugation by the IgM protease protein concentration after cell disruption, but before centrifugation (i.e. the total fraction) multiplied by 100%. No significant influence of the type of device was found, only of the applied pressure.

TABLE 1
Recovery of IgM protease antigen in supernatant

| Pressure (bar) | Recovery (%) |
|---|---|
| 100 | 5%* |
| 600 | 30% |
| 1300 | 60% |
| 2000 | 100% |

*Determined by extrapolation

### Example 3

In this example 3 the protective effect of a vaccine produced using the antigen as provided using the method of example 2 is shown.

### Study Design

Vaccines were formulated by mixing supernatant as obtained in line with Example 2 with the (oil in water) adjuvant X-Solve as available from MSD Animal Health, to arrive at a concentration of 35 μg IgM protease per ml for a first vaccine and 3.5 μg IgM protease per ml for a second vaccine, at the same amount of the oil adjuvant. For the study, thirty 3-week-old piglets were used. The piglets were allotted to three groups (different litters evenly distributed over the groups) of 10 piglets each. Group 1 and 2 were vaccinated twice intramuscularly at 3 and 5 weeks of age with the different vaccine. Group 1 received 2 ml of the first vaccine per vaccination (i.e. 70 μg of the antigen per vaccination) and Group 2 received 2 ml of the second vaccine per vaccination (i.e. 7 μg of the IgM protease antigen per dose). Group 3 was left as unvaccinated challenge control. At 7 weeks of age the piglets were transported to the challenge room and challenged immediately. There was no acclimatization period between the transport and the challenge to mimic natural stress. After challenge the pigs were observed daily for clinical signs of S. suis infection (such as depression, locomotory problems and/or neurological signs) and scored using a regular scoring system going from 0 (no signs) to 3 for severe cases. Severely affected animals were euthanized and post-mortem examined. At the end of the study (11 days after challenge) all surviving pigs were euthanized and post-mortem examined. Just before vaccination and challenge, serum blood was collected for antibody determination. At regular times before and after challenge heparin blood was collected for re-isolation of the challenge strain.

### Results

None of the vaccines induced any unacceptable site or systemic reactions and thus could be considered safe. On day of vaccination (3 weeks of age) most pigs had low or moderate maternally derived antibody titre. After vaccination, all vaccine groups showed antibody responses (data not shown). The results for the different parameters post-challenge are shown below in Table 2 (survival time in days).

TABLE 2
Post challenge data

| | Avg | Avg | # pos. | Dead after |
|---|---|---|---|---|
| Group | survival time | clinical score | blood | challenge |
| 1 | 9.4 | 14.9 | 3/10 | 3/10 |
| 2 | 8.6 | 21.5 | 3/10 | 3/10 |
| 3 | 3.4 | 64.9 | 9/10 | 8/10 |

### CONCLUSION

The results demonstrate that the vaccines induced protection in the piglets against a challenge with pathogenic Streptococcus suis 2 weeks after the second vaccination. A vaccine dose as low as 7 μg was able to provide adequate protection.

1. A method to produce a vaccine to protect a pig against a pathogenic infection with Streptococcus suis, the method comprising:
recombinantly expressing a Streptococcus suis IgM protease antigen in E. coli bacteria;
subjecting the E. coli bacteria to a high pressure homogenisation operation at a pressure of at least 500 bar to induce lysis of the E. coli bacteria and release of the IgM protease antigen into a supernatant of the lysate;
separating the supernatant from a pellet of the lysate;
mixing the supernatant comprising the IgM protease antigen with a pharmaceutically acceptable carrier to constitute the vaccine.

2. A method according to claim 1, characterised in that the pressure during the high pressure homogenisation operation is at least 1000 bar.

3. A method according to claim 1, characterised in that the pressure during the high pressure homogenisation operation is at least 1300 bar.

4. A method according to claim 1, characterised in that the pressure during the high pressure homogenisation operation is at least 2000 bar.

5. A method according to claim 1, characterised in that a device used for preforming the high pressure homogenisation operation is a French pressure cell press or a Microfluidisation device.

6. A method according to claim 1, characterised in that the IgM protease antigen is a whole IgM protease antigen.

7. A method according to claim 6, characterised in that the whole IgM protease antigen is of a Streptococcus suis bacterium of serotype 1, 2 or 7.

8. A vaccine comprising an IgM protease antigen of Streptococcus suis obtained using a method according to claim 1.

9. A vaccine according to claim 8, characterised in that the vaccine comprises at least 5% native E. coli proteins with respect to the IgM protease antigen.
